(19) United States
(12) Patent Application Publication
(10) Pub. No.: US 2005/0085780 A1
(43) Pub. Date: Apr. 21, 2005

(54) Set of compact absorbent articles

(76) Inventor: Nadia M. Corlett, Aliso Viejo, CA (US)

Correspondence Address: Eric K. Satermo;Registered Patent Agent, P.O. Box 19099, Irvine, CA, 92623-9099, US

(21) Appl. No.: 11/005,532

(22) Filed: Dec. 4, 2004

Related U.S. Application Data
(62) Division of application No. 09/560,246, filed on Apr. 26, 2000.

(51) Int. Cl. A61F 13/15; A61F 13/20

(52) U.S. Cl. 604/385.01

(57) ABSTRACT

A set of compact absorbent articles includes a plurality of absorbent articles and a packaging. Each of the absorbent articles, such as a diaper or a sanitary napkin, is reconfigurable from a normal condition to a compressed condition. The packaging includes a plurality of compartments each for receiving one of the absorbent articles in the compressed condition. Each of the absorbent articles has three dimensions, at least one of which is reduced when the absorbent article is in the compressed condition, with each of the absorbent articles being retained by the packaging when in the compressed condition.

### CROSS REFERENCE TO RELATED APPLICATION

This application is a divisional application of U.S. application Ser. No. 09/560,246 filed Apr. 26, 2004.

### BACKGROUND OF THE INVENTION

1. Field of the Invention

The present invention relates to absorbent articles such as diapers and sanitary napkins. More particularly, the present invention relates to absorbent articles that are packages at reduced volume to become ultra-compact and highly portable.

2. Description of the Related Art

One of the drawbacks of disposable diapers is portability, particularly during travel, either long-distance travel or simply to the neighborhood store. Parents need to pack a sufficient number of diapers in a diaper bag to last the duration of the trip. However, while not necessarily heavy, disposable diapers are bulky and occupy a large amount of space. Accordingly, a diaper bag in tow with traveling parents is cumbersome and a nuisance. Indeed, a single disposable diaper may not fit into most purses or a jacket breast pocket even for short trips.

In view of the foregoing, there remains a need in the art for absorbent articles that are compact and portable.

### BRIEF SUMMARY OF THE INVENTION

According to one aspect of the invention, a set of compact absorbent articles includes a plurality of absorbent articles and a packaging. Each of the absorbent articles, such as a diaper or a sanitary napkin, is reconfigurable from a normal condition to a compressed condition. The packaging includes a plurality of compartments each for receiving one of the absorbent articles in the compressed condition. Each of the absorbent articles has three dimensions, at least one of which is reduced when the absorbent article is in the compressed condition, with each of the absorbent articles being retained by the packaging when in the compressed condition.

According to various aspects of the invention, the packaging may have release seams disposed between the compartments, so that a user can remove an individual article in its compartment from the other articles in the packaging. Also, each of the absorbent articles may be are vacuumed sealed at negative pressure in the packaging. In addition, the dimension that is reduced may be reduced by at least 30% when the absorbent articles are in the compressed condition. Further, each of the absorbent articles may be reconfigured from the normal condition to the compressed condition by negative pressure, physical force, rolling, or folding.

Regarding advantages, the individually packaged compressed absorbent articles are highly compact and, accordingly, readily portable. In addition, a packaged absorbent article can be easily carried in a purse or a breast pocket of a blazer. In a multipack packaging embodiment, the multipack may be conveniently carried in, e.g., a bag, with individual absorbent articles removed from the multipack as needed.

Additional aspects, features, and advantages of the present invention will become apparent to those skilled in the art from a consideration of the following detailed description taken in conjunction with the accompanying drawings.

### BRIEF DESCRIPTION OF THE SEVERAL VIEWS OF THE DRAWINGS

FIG. 1 is a perspective schematic view of an absorbent article of the invention shown in a normal (i.e., uncompressed) condition;

FIG. 2 is a perspective schematic view of the absorbent article shown in a compressed condition;

FIG. 3 is a cross-sectional view of the absorbent article in the normal condition taken along line 3-3 of FIG. 1;

FIG. 4 is a cross-sectional view of the absorbent article in the compressed condition taken along line 4-4 of FIG. 2;

FIG. 5 is a perspective view of a reconfigured absorbent article of the invention, particularly illustrating the article in a rolled configuration;

FIG. 6 is a perspective view of a reconfigured absorbent article of the invention, particularly illustrating the article in a folded configuration;

FIG. 7 is a perspective view of the rolled absorbent article of FIG. 5, particularly illustrating the rolled article in a compressed condition;

FIG. 8 is a perspective view of the folded absorbent article of FIG. 6, particularly illustrating the folded article in a compressed condition;

FIG. 9 is a cross-sectional view of the compressed rolled absorbent article retained by packaging;

FIG. 10 is a cross-section view of the compressed folded absorbent article retained by packaging;

FIG. 11 is a schematic plan view of a plurality of compressed absorbent articles packaged together as a multipack;

FIG. 12 is a cross-sectional view of the multipack taken along line 12-12 of FIG. 11;

FIG. 13 is a cross-sectional view of a multipack of individually sealed absorbent articles folded back upon itself to be compact and portable;

FIG. 14 is a perspective view of a carrier of the invention configured to hold and retain a plurality of individually packaged compressed absorbent articles; and

FIG. 15 is a perspective view of a compressed absorbent article individually packaged in resealable packaging.

### DETAILED DESCRIPTION OF THE INVENTION

Referring more particularly to the drawings, an absorbent article is shown in FIG. 1 and generally indicated by reference numeral 10. The absorbent article 10, which may be a diaper or a sanitary napkin, is a three-dimensional object occupying a volume of space Vₙ schematically represented by the product of a length lₙ, a width wₙ, and a thickness tₙ, i.e., Vₙ=lₙ×wₙ×tₙ. The absorbent article 10 is compressible in at least one dimension but preferably in all three dimensions so that the volume of space which the article 10 occupies is reduced or compressed.

For example, the absorbent article 10 is shown in FIG. 2 with compressed length, width, and thickness dimensions l_{c}, w_{c}, and t_{c}, each of which is respectively less than the uncompressed or “normal” length, width, and thickness dimensions lₙ, wₙ, and tₙ of FIG. 1. Accordingly, a compressed volume V_{c} of the absorbent article 10 is less than the normal volume Vₙ thereof. For example, in accordance with the present invention, the absorbent article 10 is compressible such that the compressed volume V_{c} is at least one third less and preferably at least one half less than the normal volume Vₙ; that is, the compressed volume V_{c} of the absorbent article is at least 30% less and is preferably at least 50% less than the uncompressed volume Vₙ. In accordance with the principles of the present invention, the absorbent article 10 is retained in the compressed condition, for example, by vacuum packaging, thereby providing an ultra-compact and highly portable absorbent article. The absorbent article 10 retained in the compressed condition may then be easily and conveniently carried by a user until needed.

The absorbent article 10 is illustrated schematically in the drawings but may be configured as any type of absorbent article as known in the art, for example, a diaper, either infant or adult, a feminine sanitary napkin, and so on. The absorbent article 10 is made from resilient and compressible material that is able to retain fluid. Examples of diapers are disclosed in U.S. Statutory Invention Registration No. H1674 and U.S. Pat. Nos. 5,520,674; 5,522,810; 5,855,574; 5,876,393; and 5,980,500. Examples of feminine sanitary napkins are disclosed in U.S. Pat. Nos. 5,490,847; 5,653,702; 5,792,131; 5,797,894; and 6,015,934. The disclosure of each of these patents is incorporated herein by reference.

Being made from resilient absorptive material, exemplary article 10 is compressible in at least one dimension. However, to minimize the amount of compressed volume V_{c}, it is preferable for the absorbent article 10 to be compressible in three dimensions, as mentioned above. More specifically, as shown in FIG. 3, exemplary article 10 includes absorptive material 12 with inter-fiber spaces 14. To reduce at least one of the three dimensions, the absorbent article 10 is compressed, thereby substantially eliminating the inter-fiber spaces 14 and rendering the absorptive material 12 more dense, which is shown in FIG. 4.

To compress, the absorbent article 10 may be subject to compressive force, thereby forcing air out of the inter-fiber spaces 14 and compressing the absorptive material 12. Alternatively, the absorbent article 10 may be subject to vacuum, thereby drawing air out of the inter-fiber spaces 14 and compressing the absorptive material 12. Once compressed, the absorbent article 10 is retained to maintain the compact and highly portable configuration, for example, by shrink-wrap thermoplastic packaging at a vacuum or negative pressure, which will be discussed in more detail below. To use, the retaining packaging is disengaged or opened, allowing the absorbent article 10 to return to expand under the resiliency and elasticity of the absorptive material 12, which is also discussed in more detail below.

As mentioned above, the absorbent article 10 is shown in a normal condition in FIGS. 1 and 3 in which the article is unstressed, uncompressed, and ready for use. Prior to being placed in the compressed condition as shown in FIGS. 2 and 4, the absorbent article 10 may be reconfigured, for example, by rolling as schematically shown in FIG. 5 or by folding as schematically shown in FIG. 6. When reconfigured, the absorbent article 10 has at least three dimensions, for example, a width wᵣ, a thickness tᵣ, and a diameter dᵣ as shown in FIG. 5, or a length lᵣ, a width wᵣ, a thickness tᵣ, and a height hᵣ as shown in FIG. 6.

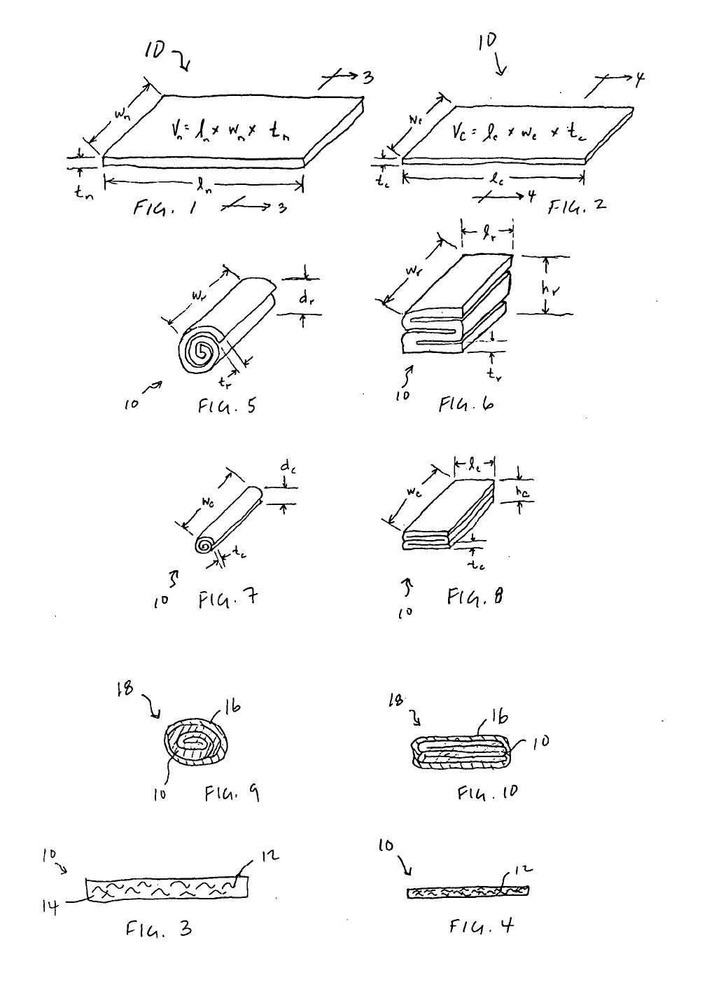

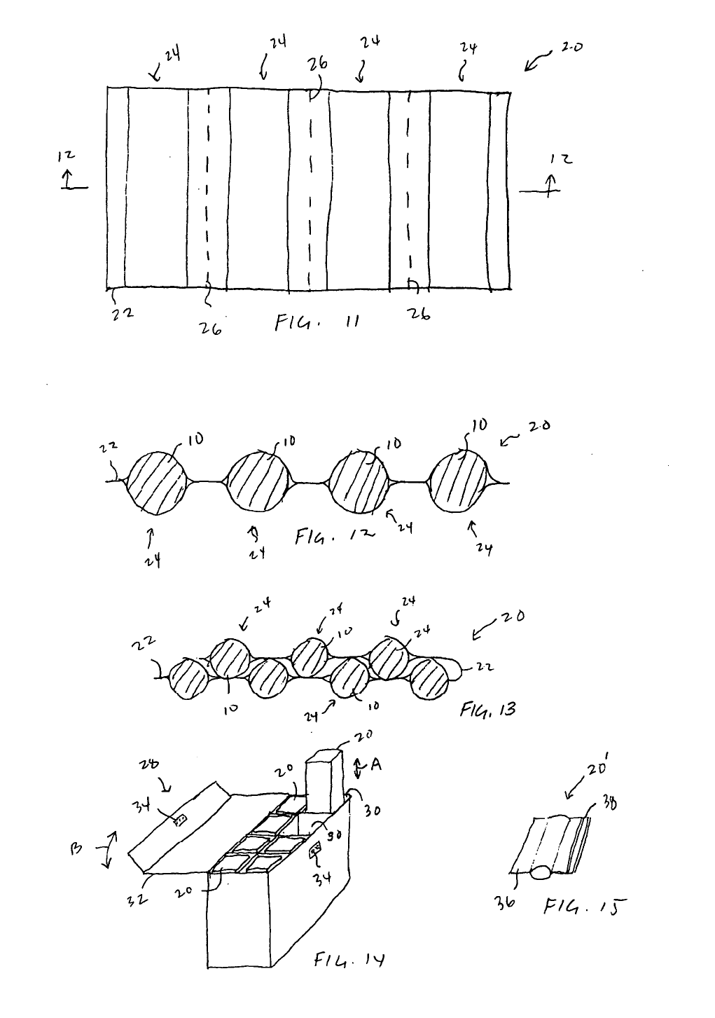

After being placed in a reconfigured condition, the absorbent article 10 may then be compressed as discussed above, thereby placing the article in a compressed condition. If reconfigured by rolling as shown in FIG. 5, then when compressed the absorbent article 10 takes on the compressed condition shown in FIG. 7 with compressed dimensions of a width w_{c}, a thickness t_{c}, and a diameter d_{c}. If reconfigured by folding as shown in FIG. 6, then when compressed the absorbent article 10 takes on the compressed condition shown in FIG. 8 with compressed dimensions of a length l_{c}, a width w_{c}, a thickness t_{c}, and a height h_{c}. Analogous to the description above in relation to FIGS. 1 and 2, at least one of the compressed dimensions but preferably all of the compressed dimensions of the absorbent article 10 are respectively less than the reconfigured dimensions thereof.

After being compressed, the absorbent article 10 may then be retained to maintain the compressed condition, for example, with packaging 16 as shown in FIGS. 9 and 10, thereby yielding an individually packaged article 18. As mentioned above, the packaging 16 may be a thermoplastic material that allows the compressed absorbent article 10 to be hermetically sealed therein. Alternatively, the packaging 16 may be any other material suitable for retaining the absorbent article 10 in the compressed condition. The packaging 16 ensures that the absorbent article 10 is clean and sanitary when the packaged article 18 is opened for use.

In addition to individually packaged absorbent articles 18, a plurality of compressed absorbent articles 10 may be packaged together. This multipack of compressed absorbent articles 10 is shown in FIGS. 11 and 12 and indicated by reference numeral 20. The multipack 20 of articles includes packaging 22 for retaining a plurality of absorbent articles 10 in the compressed condition. The absorbent articles 10 are configured within the packaging 22 such that each article 10 is individually sealed, which is particularly shown in FIG. 12 and indicated by reference numeral 24. The packaging 22 may include perforations 26 between the individually sealed articles 24 so that a user may remove articles 24 as needed. In addition, the packaging 22 may be substantially flexible so that the multipack 20 may be folded back onto itself to be more compact and portable as shown in FIG. 13.

As mentioned above, the compressed absorbent article 10 may be packaged individually as shown in FIGS. 9 and 10. In accordance with the present invention, individually packaged articles 20 may be arranged in a carrier 28 for convenient transport. Exemplary carrier 28 includes a plurality of compartments 30 each configured to receive a packaged article 20 as shown by arrow A. The carrier 28 may also include a hinged cover 32 which pivots as shown by arrow B. The carrier 28 may also include releasable fastening means 34 such as complementary hook-and-eye fasteners 34 for retaining the cover 32 in a closed condition on the carrier.

Individually packaged compressed absorbent articles 20 may include sealable packaging 36 as shown in FIG. 15. More specifically, exemplary packaging 36 may include a resealable closure mechanism 38. Accordingly, a user may open the packaging 36 and remove the absorbent article 10, thereby allowing the compressed absorbent article to regain the normal condition for use. The now-empty packaging 36 may now receive and store a soiled absorbent article to be discarded in the future.

Those skilled in the art will understand that the preceding exemplary embodiments of the present invention provide the foundation for numerous alternatives and modifications thereto. These and other modifications are also within the scope of the present invention. Accordingly, the present invention is not limited to that precisely as shown and described above but by the scope of the appended claims.

1. A set of compact absorbent articles, the set comprising:
a plurality of absorbent articles each reconfigurable from a normal condition to a compressed condition; and
a packaging including a plurality of compartments each for receiving one of the absorbent articles in the compressed condition; and
each of the absorbent articles having three dimensions at least one of which is reduced when the absorbent article is in the compressed condition; and
each of the absorbent articles being retained by the packaging when in the compressed condition.

2. A set as claimed in claim 1 wherein the packaging includes release seams disposed between the compartments.

3. A set as claimed in claim 1 wherein each of the absorbent articles are vacuumed sealed at negative pressure in the packaging.

4. A set as claimed in claim 1 wherein the at least one of the dimensions is reduced by at least 30% when the absorbent articles are in the compressed condition.

5. A set as claimed in claim 1 wherein each of the absorbent article occupies a volume of space, the volume of space being reduced by at least about 30% when each of the absorbent articles is reconfigured from the normal condition to the compressed condition.

6. A set as claimed in claim 1 wherein each of the absorbent articles is reconfigured from the normal condition to the compressed condition by negative pressure.

7. A set as claimed in claim 1 wherein each of the absorbent articles is reconfigured from the normal condition to the compressed condition by physical force.

8. A set as claimed in claim 1 wherein each of the absorbent articles is reconfigured from the normal condition to the compressed condition by rolling.

9. A set as claimed in claim 1 wherein each of the absorbent articles is reconfigured from the normal condition to the compressed condition by folding.

10. A set as claimed in claim 1 wherein each of the absorbent articles is a diaper.

11. A set as claimed in claim 1 wherein each of the absorbent articles is a sanitary napkin.
